(19) United States
(12) Patent Application Publication
(10) Pub. No.: US 2020/0056154 A1
(43) Pub. Date: Feb. 20, 2020

(54) CELL CULTURE SUBSTRATE HAVING TWO ACRYLATE STRUCTURAL UNITS

(71) Applicant: TERUMO KABUSHIKI KAISHA, Tokyo (JP)

(72) Inventors: Takao ANZAI, KANAGAWA (JP); Ichiro HIRAHARA, KANAGAWA (JP)

(73) Assignee: TERUMO KABUSHIKI KAISHA, Tokyo (JP)

(21) Appl. No.: 16/541,938

(22) Filed: Aug. 15, 2019

(30) Foreign Application Priority Data
Aug. 16, 2018 (JP) 2018-153272

(51) Int. Cl. C12N 5/0775

(52) U.S. Cl. CPC C12N 5/0667; C12N 2533/00

(57) ABSTRACT

The invention is to provide a means capable of achieving both cellular adhesiveness and antithrombogenicity with balance. Provided is a cell culture substrate comprising a coating layer on at least one side of a polymer substrate, wherein the coating layer includes a copolymer having 5 to 65% by mole of a structural unit (1) derived from alkoxyalkyl (meth)acrylate of Formula (1) and 95 to 35% by mole of a structural unit (2) derived from furfuryl (meth)acrylate of Formula (2) (the total of the structural unit (1) and the structural unit (2) is 100% by mole).

### TECHNICAL FIELD

The present invention relates to a cell culture substrate excellent in cellular adhesiveness, and a bioreactor and a method for culturing a stem cell using the cell culture substrate.

### BACKGROUND

In recent years, a cell culture technology has been used in the development of regenerative medicine or drug discovery. In particular, attention has been paid to use of stem cells, and technology for repairing and replacing damaged or defective tissues has been actively studied by using stem cells expanded from donor cells. Most of cells of animals including humans are adherent (scaffold-dependent) cells which cannot survive in a floating state and survive in a state of being adhered to something. For this reason, various developments of functional culture substrates for culturing adherent (scaffold-dependent) cells at high density to obtain cultured tissues similar to living tissues have been conducted.

As a cell culture substrate, plastic (for example, polystyrene) or glass vessels have been conventionally used, and it has been reported that a plasma treatment or the like is performed to a surface of these cell vessels. A substrate subjected to the treatment has excellent adhesion to cells, and can be used to grow cells and maintain their function.

Meanwhile, regarding a structure of the cell culture substrate (cell culture vessel), in addition to a conventional flat dish (plate) structure, various structures, such as a structure in which a porous body is inserted as a culture scaffold in a bag, a hollow fiber structure, a sponge structure, a flocculent (glass wool) structure, and a structure in which a plurality of dishes are laminated, have been developed. It is difficult or impossible to perform plasma irradiation to culture vessels having such diversified and complicated structures.

In this regard, a technique using a polymer that has adhesiveness to cells (cellular adhesiveness) and a property to prompt proliferation of cells (cell proliferation activity) has been proposed. For example, Non Patent Literature 1 discloses that a PET substrate covered with a homopolymer of tetrahydrofurfuryl acrylate (PTHFA; polytetrahydrofurfuryl acrylate) and a homopolymer of methoxyethyl acrylate (PMEA; polymethoxyethyl acrylate) has cellular adhesiveness with respect to umbilical vein endothelial cells or aortic endothelial cells but does not adhere to platelets.

### CITATION LIST

### Non Patent Literatures

Non Patent Literature 1: Colloids and Surfaces B; Biointerfaces 145 (2016) 586-596

### SUMMARY OF THE INVENTION

As disclosed in the Non Patent Literature 1, polymethoxyethyl acrylate (PMEA) and polytetrahydrofurfuryl acrylate (PTHFA) can impart cellular adhesiveness and antithrombogenicity to a cell culture substrate, as compared to a PET substrate which is not covered with a polymer. Further, since such a polymer is excellent in coating operability, even in the case of a cell culture substrate having a complicated structure as described above, the polymer can provide cellular adhesiveness.

On the other hand, in recent years, in culturing of stem cells or development of artificial blood vessels, higher cellular adhesiveness and antithrombogenicity are demanded, and with the polymers as described above, both characteristics cannot be satisfied at the same time.

Therefore, the present invention has been made in view of the above-described circumstances, and an object thereof is to provide a means capable of achieving both cellular adhesiveness and antithrombogenicity with balance in a technique of coating a cell culture substrate (cell culture vessel) using a polymer.

The present inventors have conducted intensive studies to solve the above-described problems. As a result, the present inventors have found that the problems can be solved by coating a surface of a cell culture substrate (polymer substrate) using an alkoxyalkyl (meth)acrylate-furfuryl (meth)acrylate copolymer having a specific composition and structure. The present invention has been completed on the basis of the above finding.

That is, the object can be achieved by a cell culture substrate (substrate for cell culture) comprising a coating layer on at least one side of a polymer substrate, wherein the coating layer contains a copolymer having 5 to 65% by mole of a structural unit (1) derived from alkoxyalkyl (meth)acrylate of the following Formula (1) and 95 to 35% by mole of a structural unit (2) derived from furfuryl (meth)acrylate of the following Formula (2) (the total of the structural unit (1) and the structural unit (2) is 100% by mole).

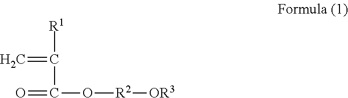

In the Formula (1), R¹ represents a hydrogen atom or a methyl group, R² represents an alkylene group having 2 or 3 carbon atoms, and R³ represents an alkyl group having 1 to 3 carbon atoms;

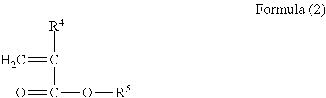

in the Formula (2), R⁴ represents a hydrogen atom or a methyl group, and R⁵ represents a group represented by the following Formula (2-1) or the following Formula (2-2):

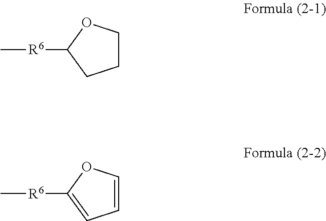

in the Formula (2-1) and the Formula (2-2), R⁶ represents an alkylene group having 1 to 3 carbon atoms.

### BRIEF DESCRIPTION OF DRAWINGS

FIG. 1 is a partial side view illustrating an embodiment of a bioreactor (hollow fiber type bioreactor) of the present invention.

FIG. 2 is a partially cut-away side view of the bioreactor of FIG. 1.

### DETAILED DESCRIPTION

The cell culture substrate (substrate for cell culture) of the present invention has a coating layer on at least one side of a polymer substrate, wherein the coating layer includes a copolymer comprising 5 to 65% by mole of a structural unit (1) derived from alkoxyalkyl (meth)acrylate of the following Formula (1) and 95 to 35% by mole of a structural unit (2) derived from furfuryl (meth)acrylate of the following Formula (2) (the total of the structural unit (1) and the structural unit (2) is 100% by mole).

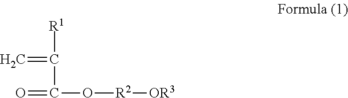

wherein R¹ represents a hydrogen atom or a methyl group, R² represents an alkylene group having 2 or 3 carbon atoms, and R³ represents an alkyl group having 1 to 3 carbon atoms;

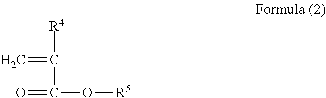

wherein R⁴ represents a hydrogen atom or a methyl group, and R⁵ represents a group represented by the following Formula (2-1) or the following Formula (2-2):

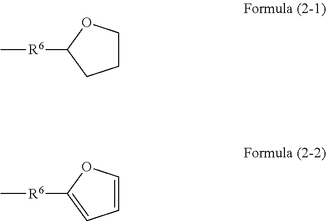

wherein R⁶ represents an alkylene group having 1 to 3 carbon atoms.

By the copolymer according to the present invention, it is possible to provide a cell culture substrate (cell culture vessel) having both cellular adhesiveness and antithrombogenicity in a well-balanced manner.

In the present description, the alkoxyalkyl (meth)acrylate of the Formula (1) is simply referred to as “alkoxyalkyl (meth)acrylate”. Further, the structural unit (1) derived from alkoxyalkyl (meth)acrylate of the Formula (1) is simply referred to as “alkoxyalkyl (meth)acrylate structural unit” or “structural unit (1)”. Further, the furfuryl (meth)acrylate of the above Formula (2) is simply referred to as “furfuryl (meth)acrylate”. Further, the structural unit (2) derived from furfuryl (meth)acrylate of the above Formula (2) is simply referred to as “furfuryl (meth)acrylate structural unit” or “structural unit (2)”. Furthermore, the copolymer having the structural unit (1) and the structural unit (2) is simply referred to as “copolymer” or “copolymer according to the present invention.”

Further, in the present description, the term “(meth)acrylate” includes both acrylate and methacrylate”. Similarly, the term “(meth)acrylic acid” includes both acrylic acid and methacrylic acid, and “(meth)acryloyl” includes both acryloyl and methacryloyl.

The cell culture substrate of the present invention has a feature in that a coating layer containing the copolymer according to the present invention is formed on at least one surface of the polymer substrate. The coating film (coating layer) formed by using the copolymer has cellular adhesiveness and antithrombogenicity with excellent balance. Further, the coating film (coating layer) formed by using the copolymer is also excellent in cell proliferation activity (cell expanding ability). Here, the mechanism for exhibiting the effects by the present invention is presumed to be as follows. Incidentally, the present invention is not limited to the following presumption.

Conventionally, as a means for imparting cell adhesion, there has been a method of applying a cell adhesion factor such as fibronectin, laminin, or collagen to a substrate, a method of subjecting a substrate to treatment with plasma, gamma rays, or electrons, and the like. Of them, the former method has problems in that a cell adhesion factor is expensive and cannot be typically reused since the cell adhesion factor is a natural material, and the like. Further, in the latter method, the plasma treatment can impart particularly excellent cell adhesion to a substrate. Meanwhile, in recent years, a structure in which a porous body is inserted as a culture scaffold in a bag, a hollow fiber structure, a sponge structure, a flocculent (glass wool) structure, and a structure in which a plurality of dishes are laminated are used as a suitable culture scaffold. However, the latter method has a problem in that it is difficult or impossible to apply the method to such diversified and complicated structure. Currently, from the viewpoint that the complicated structures are excellent as a culture scaffold, there is a need for a means to provide a culture scaffold with cell adhesion comparable to or greater than that by plasma treatment.

Non Patent Literature 1 discloses that a coating film of polytetrahydrofurfuryl acrylate (PTHFA) or polymethoxyethyl acrylate (PMEA) have cellular adhesiveness and antithrombogenicity. The coating film of polytetrahydrofurfuryl acrylate (PTHFA) is certainly excellent in cellular adhesiveness. However, in order to meet a demand for more efficiently recovering desired cells, much higher cellular adhesiveness is demanded. Further, as a result of studies by the present inventors, it is hard to say that the coating film of polytetrahydrofurfuryl acrylate (PTHFA) has sufficient antithrombogenicity depending on its applications (for example, a bioreactor or an artificial blood vessel). Further, the coating film of polymethoxyethyl acrylate (PMEA) has certainly excellent antithrombogenicity but is greatly inferior to polytetrahydrofurfuryl acrylate (PTHFA) in cellular adhesiveness, and thus, it is hard to say that the coating film of polymethoxyethyl acrylate (PMEA) has sufficient cellular adhesiveness depending on its applications (for example, a bioreactor or an artificial blood vessel). As such, development of a polymer having excellent cellular adhesiveness and antithrombogenicity with balance has been demanded, but under present circumstances, such a polymer has not been obtained.

In view of the above-described circumstances, the present inventors have evaluated copolymers of alkoxyalkyl (meth)acrylate and furfuryl (meth)acrylate for cellular adhesiveness and antithrombogenicity, and as a result, have first found that cellular adhesiveness and antithrombogenicity can be exhibited with balance in a specific composition. Further, the present inventors have also found that these copolymers are excellent in cell proliferation activity (cell expansion ability).

The detailed mechanism thereof is not clear but is presumed as follows. That is, although the structural unit derived from alkoxyalkyl (meth)acrylate can provide excellent antithrombogenicity to a substrate, regarding a coating film of a homopolymer of alkoxyalkyl (meth)acrylate, cells are adhered but are difficult to expand and proliferously grow so that the effect of providing excellent cellular adhesiveness/proliferation activity to a substrate is inferior. On the other hand, regarding a coating film of a homopolymer of furfuryl (meth)acrylate, since adhered cells are easy to expand and proliferously grow, excellent cellular adhesiveness/proliferation activity can be provided to a substrate but antithrombogenicity is inferior. However, it is presumed that when the composition of the structural unit derived from furfuryl (meth)acrylate is set to 95 to 35% by mole, a contact surface between a cell and a substrate is increased, to promote expansion and proliferation of adhered cells, while when the composition of the structural unit derived from alkoxyalkyl (meth)acrylate is set to 5 to 65% by mole, moderate antithrombogenicity (effect of suppressing or preventing adhesion/attachment of platelets, and effect of suppressing or preventing activation of platelets) is provided, and the coating film (coating layer) formed by the copolymer according to the present invention can attained balance between cellular adhesiveness (and further cell proliferation activity) and antithrombogenicity. As a result, the coating layer containing the copolymer according to the present invention can exhibit moderate antithrombogenicity (effect of suppressing or preventing adhesion/attachment of platelets and effect of suppressing or preventing activation of platelets), particularly, effect of suppressing or preventing adhesion/attachment of platelets, and can exhibit excellent cellular adhesiveness and further cell proliferation activity (cell expanding ability). Incidentally, in the following Examples, it is shown that the coating film of the homopolymer of methoxyethyl acrylate has only a little more than 50% of cellular adhesiveness as compared to the coating film of the homopolymer of tetrahydrofurfuryl acrylate (comparison between Comparative Example 6 and Comparative Example 9 in the following Table 1). On the other hand, it is shown that the coating film of the copolymer of tetrahydrofurfuryl acrylate and methoxyethyl acrylate as an example of the copolymer according to the present invention can significantly improve cellular adhesiveness as compared to the coating film of the homopolymer of tetrahydrofurfuryl acrylate (comparison between Examples 5 to 8 and Comparative Example 6 in the following Table 1). In view that it is generally considered that cellular adhesiveness of a coating film of a copolymer of a certain monomer A and a monomer B inferior in cellular adhesiveness is degraded as compared to a coating film of a homopolymer of the monomer A, the above-described result is very surprising.

Furthermore, according to the present invention, the coating layer can be formed by applying a solution obtained by dissolving the copolymer in a solvent to a surface of a polymer substrate. Therefore, the coating layer (cell adhesion layer) can be easily formed even with respect to a cell culture substrate (cell culture vessel) having various shapes or designs.

Therefore, the cell culture substrate of the present invention can prevent adhesion/attachment of blood components (particularly, platelets) and selectively adhere or culture desired cells (particularly, stem cells) even in the case of using samples containing blood components.

Hereinafter, a preferred embodiment of the present invention will be described. Incidentally, the present invention is not limited only to the following embodiment.

In the present description, the term “X to Y” which indicates a range means the term “X or more and Y or less” including X and Y. Further, unless otherwise specified, operations and measurements of physical properties and the like are conducted under conditions of room temperature (20 to 25° C.)/relative humidity of 40 to 50% RH.

### <Cell Culture Substrate>

The cell culture substrate of the present invention comprises a coating layer containing the copolymer formed on at least one surface of a polymer substrate. The coating layer containing the copolymer according to the present invention can exhibit cellular adhesiveness and antithrombogenicity with balance. Further, the coating layer containing the copolymer according to the present invention is also excellent in cell expansion activity (cell proliferation activity). In addition, the coating layer can be simply formed in such a manner that the copolymer is dissolved in a solvent and the resultant solution is applied to a surface of the polymer substrate. Therefore, by using the copolymer according to the present invention, a coating layer (cell adhesion layer) providing antithrombogenicity and cellular adhesiveness (and further cell proliferation activity) with balance can be formed on a surface of cell culture substrate (cell culture vessel) regardless of it shape or design.

### [Copolymer]

The copolymer according to the present invention has 5 to 65% by mole of a structural unit (1) derived from alkoxyalkyl (meth)acrylate represented by Formula (1) and 95 to 35% by mole of a structural unit (2) derived from furfuryl (meth)acrylate represented by Formula (2). Herein, the total of the structural unit (1) and the structural unit (2) is 100% by mole.

The copolymer has the structural unit (1), the structural unit (2), and as necessary, a structural unit derived from another monomer which will be described later in detail. Here, the arrangement of each structural unit is not particularly limited, but may be in the form of block (block copolymer), random (random copolymer), or alternate (alternate copolymer).

The alkoxyalkyl (meth)acrylate (structural unit (1)) imparts antithrombogenicity to a substrate. The furfuryl (meth)acrylate (structural unit (2)) imparts cellular adhesiveness to the substrate. In addition, the furfuryl (meth)acrylate (structural unit (2)) is presumed to impart cell expansion ability (cell proliferation activity) to the substrate. In particular, by combining the alkoxyalkyl (meth)acrylate (structural unit (1)) and the furfuryl (meth)acrylate (structural unit (2)) at a specific ratio, cellular adhesiveness can be significantly improved (there is a synergetic effect) as compared to a homopolymer of alkoxyalkyl (meth)acrylate or a homopolymer of furfuryl (meth)acrylate. In addition thereto, by applying a solution of the copolymer to a surface of a polymer substrate, a coating layer can be simply formed on a substrate having various shapes. Therefore, with the copolymer according to the present invention, a coating layer (cell adhesion layer) having excellent antithrombogenicity and cellular adhesiveness (and further cell proliferation activity) can be formed on a cell culture substrate (cell culture vessel) having various shapes or designs.

The structural unit (1) constituting the copolymer according to the present invention is contained at a ratio of 5 to 65% by mole with respect to the total (100% by mole) of the structural unit (1) and the structural unit (2), and the structural unit (2) is contained at a ratio of 95 to 35% by mole with respect to the total (100% by mole) of the structural unit (1) and the structural unit (2). Herein, when the composition of the structural unit (1) is less than 5% by mole, the effect (antithrombogenicity) by the alkoxyalkyl (meth)acrylate (structural unit (1)) cannot be exhibited (see Comparative Example 16 in the following Table 2). On the other than, when the composition of the structural unit (1) is more than 65% by mole, the effect(s) (cellular adhesiveness promoting (improving) effect and further cell proliferation activity providing effect) by the furfuryl (meth)acrylate (structural unit (2)) cannot be exhibited, and cellular adhesiveness would be degraded as compared to a homopolymer of furfuryl (meth)acrylate (see Comparative Examples 7 to 9 in the following Table 1).

From the viewpoint of further improving the balance between cellular adhesiveness (and further cell proliferation activity) and antithrombogenicity, or the like, it is preferable that the structural unit (1) is contained at a ratio of 10 to 60% by mole with respect to the total of the structural unit (1) and the structural unit (2), and the structural unit (2) is contained at a ratio of 90 to 40% by mole with respect to the total of the structural unit (1) and the structural unit (2). More preferably, the structural unit (1) is contained at a ratio of 30 to 60% by mole with respect to the total of the structural unit (1) and the structural unit (2), and the structural unit (2) is contained at a ratio of 70 to 40% by mole with respect to the total of the structural unit (1) and the structural unit (2). Particularly preferably, the structural unit (1) is contained at a ratio of 40 to 60% by mole with respect to the total of the structural unit (1) and the structural unit (2), and the structural unit (2) is contained at a ratio of 60 to 40% by mole with respect to the total of the structural unit (1) and the structural unit (2). That is, according to the preferred embodiment of the present invention, the copolymer is a copolymer having 10 to 60% by mole of the structural unit (1) derived from alkoxyalkyl (meth)acrylate of Formula (1) and 90 to 40% by mole of the structural unit (2) derived from furfuryl (meth)acrylate of Formula (2) (the total of the structural unit (1) and the structural unit (2) is 100% by mole). According to the more preferred embodiment of the present invention, the copolymer is a copolymer having 30 to 60% by mole of the structural unit (1) derived from alkoxyalkyl (meth)acrylate of Formula (1) and 70 to 40% by mole of the structural unit (2) derived from furfuryl (meth)acrylate of Formula (2) (the total of the structural unit (1) and the structural unit (2) is 100% by mole). According to the particularly preferred embodiment of the present invention, the copolymer is a copolymer having 40 to 60% by mole of the structural unit (1) derived from alkoxyalkyl (meth)acrylate of Formula (1) and 60 to 40% by mole of the structural unit (2) derived from furfuryl (meth)acrylate of Formula (2) (the total of the structural unit (1) and the structural unit (2) is 100% by mole).

The copolymer according to the present invention essentially includes the structural unit (1) and the structural unit (2), but may further have a structural unit derived from another monomer in addition to the structural unit (1) and the structural unit (2). Here, the another monomer is not particularly limited as long as it does not inhibit desired characteristics (antithrombogenicity, cell adhesion and/or cell proliferation activity). Specific examples of the another monomer include acrylamide, N,N-dimethylacrylamide, N,N-diethylacrylamide, methacrylamide, N,N-dimethylmethacrylamide, N,N-diethylmethacrylamide, ethylene, propylene, N-vinylacetamide, N-isopropenyl acetamide, N-(meth)acryloyl morpholine, and the like. A composition of the structural unit derived from another monomer in the case where the copolymer further has a structural unit derived from the another monomer is not particularly limited as long as it does not inhibit desired characteristics (antithrombogenicity, cell adhesion and cell proliferation activity), but the composition of the structural unit derived from another monomer is preferably more than 0% by mole and less than 10% by mole and more preferably about 3 to 8% by mole, with respect to the total of the structural unit (1) and the structural unit (2).

For the purpose of improving antithrombogenicity and cell adhesion (and further cell proliferation activity), it is preferable that the copolymer includes no structural units derived from another monomer, that is, the copolymer according to the present invention is formed only of the structural unit (1) and the structural unit (2). That is, according to the preferred embodiment of the present invention, the copolymer is composed of the structural unit (1) and the structural unit (2). Therefore, according to the more preferred embodiment of the present invention, the copolymer is a copolymer composed of 10 to 60% by mole of the structural unit (1) and 90 to 40% by mole of the structural unit (2) (the total of the structural unit (1) and the structural unit (2) is 100% by mole). A coating film of the copolymer having such a composition can show antithrombogenicity and cellular adhesiveness (and further cell proliferation activity) with better balance. According to the further more preferred embodiment of the present invention, the copolymer is a copolymer composed of 30 to 60% by mole of the structural unit (1) and 70 to 40% by mole of the structural unit (2) (the total of the structural unit (1) and the structural unit (2) is 100% by mole). A coating film of the copolymer having such a composition has antithrombogenicity and cellular adhesiveness (and further cell proliferation activity) with further better balance.

According to the particularly preferred embodiment of the present invention, the copolymer is a copolymer composed of 40 to 60% by mole of the structural unit (1) and 60 to 40% by mole of the structural unit (2) (the total of the structural unit (1) and the structural unit (2) is 100% by mole). A coating film of the copolymer having such a composition can further improve antithrombogenicity.

The structural unit (1) is derived from alkoxyalkyl (meth)acrylate of the following Formula (1). The structural unit (1) constituting the copolymer may be used singly or in combination of two or more kinds. That is, the structural unit (1) may be composed only of sole structural unit derived from alkoxyalkyl (meth)acrylate of the following Formula (1), or may be composed of two or more structural units derived from alkoxyalkyl (meth)acrylate of the following Formula (1). In the latter case, each structural unit may be present in the form of block or random. Further, when the structural unit (1) is composed of two or more structural units derived from alkoxyalkyl (meth)acrylate of the following Formula (1), a composition of the structural unit (1) is a total ratio (molar ratio (% by mole)) of the structural units derived from alkoxyalkyl (meth)acrylate with respect to the total of the structural unit (1) and the structural unit (2).

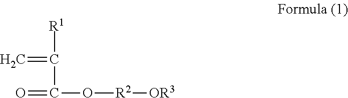

In the Formula (1), R¹ is a hydrogen atom or a methyl group. R² is an alkylene group having 2 or 3 carbon atoms. The alkylene group having 2 or 3 carbon atoms includes an ethylene group (—CH₂CH₂—), a trimethylene group (—CH₂CH₂CH₂—), and a propylene group (—CH(CH₃)CH₂— or —CH₂CH(CH₃)—). Among these, from the viewpoint of further improvement in antithrombogenicity, more favorable balance between antithrombogenicity and cellular adhesiveness (and further cell proliferation activity), and the like, R² preferably represents an ethylene group (—CH₂CH₂—) or a propylene group, and more preferably an ethylene group (—CH₂CH₂—). Further, R³ is an alkyl group having 1 to 3 carbon atoms. The alkyl group having 1 to 3 carbon atoms includes a methyl group, an ethyl group, a n-propyl group, and an isopropyl group. Among these, from the viewpoint of more favorable balance between antithrombogenicity and cellular adhesiveness (and further cell proliferation activity), and the like, R³ preferably represents a methyl group or an ethyl group, and more preferably a methyl group.

Specifically, examples of the alkoxyalkyl (meth)acrylate include methoxymethyl acrylate, methoxyethyl acrylate, methoxypropyl acrylate, ethoxymethyl acrylate, ethoxyethyl acrylate, ethoxypropyl acrylate, ethoxybutyl acrylate, propoxymethyl acrylate, butoxyethyl acrylate, methoxybutyl acrylate, methoxymethyl methacrylate, methoxyethyl methacrylate, ethoxymethyl methacrylate, ethoxyethyl methacrylate, propoxymethyl methacrylate, butoxyethyl methacrylate, and the like. Among these, from the viewpoint of further improving cell adhesion (and further cell proliferation activity), or the like, methoxyethyl (meth)acrylate, ethoxyethyl (meth)acrylate, or methoxybutyl (meth)acrylate is preferable, methoxyethyl (meth)acrylate is more preferable, and methoxyethyl acrylate (MEA) is particularly preferable.

The structural unit (2) is derived from furfuryl (meth)acrylate of the following Formula (2). The structural unit (2) constituting the copolymer may be used singly or in combination of two or more kinds. That is, the structural unit (2) may be composed only of sole structural unit derived from furfuryl (meth)acrylate of the following Formula (2), or may be composed of two or more structural units derived from furfuryl (meth)acrylate of the following Formula (2). In the latter case, each structural unit may be present in the form of block or random. Further, when the structural unit (2) is composed of two or more structural units derived from furfuryl (meth)acrylate of the following Formula (2), a composition of the structural unit (2) is a total ratio (molar ratio (% by mole)) of the structural units derived from furfuryl (meth)acrylate with respect to the total of the structural unit (1) and the structural unit (2).

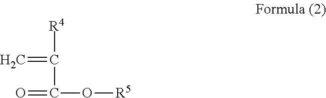

In the above Formula (2), R⁴ is a hydrogen atom or a methyl group.

Further, R⁵ is a group represented by the following Formula (2-1) or the following Formula (2-2):

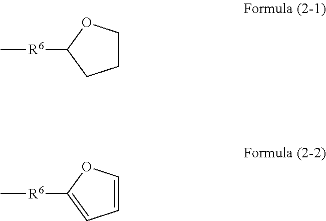

Among these, from the viewpoint of further improvement in cellular adhesiveness (and further cell proliferation activity), more favorable balance between antithrombogenicity and cellular adhesiveness (and further cell proliferation activity), and the like, R⁵ preferably represents the group represented by the Formula (2-1).

In the Formula (2-1) and the Formula (2-2), R⁶ is an alkylene group having 1 to 3 carbon atoms. The alkylene group having 1 to 3 carbon atoms includes a methylene group (—CH₂—), an ethylene group (—CH₂CH₂—), a trimethylene group (—CH₂CH₂CH₂—), and a propylene group (—CH(CH₃)CH₂— or —CH₂CH(CH₃)—). Among these, from the viewpoint of further improvement in cellular adhesiveness (and further cell proliferation activity), more favorable balance between antithrombogenicity and cellular adhesiveness (and further cell proliferation activity), and the like, R⁵ preferably represents a methylene group (—CH₂—) or an ethylene group (—CH₂CH₂—), and more preferably a methylene group (—CH₂—).

Specifically, examples of the furfuryl (meth)acrylate include tetrahydrofurfuryl acrylate, 5-[2-(acryloyloxy)ethyl]tetrahydrofuran, 2-furanylmethyl acrylate, 5-[2-(acryloyloxy)ethyl]furane, tetrahydrofurfuryl methacrylate, 5-[2-(methacryloyloxy)ethyl]tetrahydrofuran, 2-furanylmethyl methacrylate, 5-[2-(methacryloyloxy)ethyl]furane, and the like. Among these, from the viewpoint of further improvement in cellular adhesiveness (and further cell proliferation activity), and the like, tetrahydrofurfuryl (meth)acrylate is preferred, and tetrahydrofurfuryl acrylate (THFA) is more preferred.

A weight average molecular weight (Mw) of the copolymer according to the present invention is not particularly limited, but is preferably 50,000 to 1,000,000. Within the above range, the solubility of the copolymer in a solvent can be improved and application to a substrate can be uniformly conducted with ease. From the viewpoint of improving coating film formability, the weight average molecular weight of the copolymer is more preferably 100,000 to 500,000 and particularly preferably 250,000 to 400,000. In the present description, as the “weight average molecular weight (Mw),” a value measured by gel permeation chromatography (GPC) using polystyrene as a standard and tetrahydrofuran (THF) as a mobile phase respectively is adopted. Specifically, the copolymer is dissolved in tetrahydrofuran (THF) so as to have a concentration of 10 mg/ml, thereby preparing a sample. Regarding the sample prepared as above, GPC column LF-804 (manufactured by Showa Denko K.K.) is attached to a GPC system LC-20 (manufactured by SHIMADZU CORPORATION), THF is supplied as a mobile phase, and polystyrene is used as a standard, to measure GPC of the copolymer. After preparing a calibration curve with polystyrene as standards, the weight average molecular weight (Mw) of the copolymer is calculated on the basis of the curve.

The copolymer according to the present invention can be produced by employing a conventionally known polymerization method such as bulk polymerization, suspension polymerization, emulsion polymerization, solution polymerization, living radical polymerization method, polymerization method using a macroinitiator, polycondensation method, or the like, for example, although not particularly limited thereto. Specifically, in a case where the copolymer according to the present invention is a block copolymer, for example, a living radical polymerization method or a polymerization method using a macroinitiator is preferably used. As the living radical polymerization method, although not particularly limited thereto, a method described in JP H11-263819 A, JP 2002-145971 A, JP 2006-316169 A, or the like, an atom transfer radical polymerization (ATRP) method, and the like can be applied similarly or appropriately modified, for example.

Alternatively, for example, in a case where the copolymer according to the present invention is a random copolymer, it is preferable to use a method of stirring the alkoxyalkyl (meth)acrylate of the Formula (1), furfuryl (meth)acrylate of the above Formula (2), and as necessary, one or two or more kinds of monomer which is copolymerizable with those components (another monomer, copolymerizable monomer), in a polymerization solvent, with a polymerization initiator to prepare a monomer solution, and heating the monomer solution to perform copolymerization. In the method, a polymerization solvent which can be used in the preparation of the monomer solution is not particularly limited as long as it can dissolve the monomer used above. Examples thereof include aqueous solvents such as water, alcohols such as methanol, ethanol, propanol, and isopropanol, and polyethylene glycols; aromatic solvents such as toluene, xylene, and tetralin; halogen-based solvents such as chloroform, dichloroethane, chlorobenzene, dichlorobenzene, and trichlorobenzene; and the like. Among these, taking in consideration of easy dissolution of the monomer, or the like, methanol is preferable. Further, a concentration of the monomer in the monomer solution is not particularly limited, but the concentration of the monomer in the monomer solution is typically 15 to 60% by weight, more preferably 20 to 50% by weight, and particularly preferably 25 to 45% by weight. Incidentally, the concentration of the monomer means the total concentration of the alkoxyalkyl (meth)acrylate of the above Formula (1), the furfuryl (meth)acrylate of the above Formula (2), and if being used, a monomer which is copolymerizable with those components (another monomer, copolymerizable monomer).

The polymerization initiator is not particularly limited, and a known polymerization initiator may be used. From the viewpoint of high polymerization stability, the polymerization initiator is preferably a radical polymerization initiator. Specific examples thereof include persulfates such as potassium persulfate (KPS), sodium persulfate, and ammonium persulfate; peroxides such as hydrogen peroxide, t-butyl peroxide, and methyl ethyl ketone peroxide; and azo compounds such as azobisisobutyronitrile (AIBN), 2,2′-azobis(4-methoxy-2,4-dimethylvaleronitrile), 2,2′-azobis(2,4-dimethylvaleronitrile), 2,2′-azobis[2-(2-imidazoline-2-yl)propane]dihydrochloride, 2,2′-azobis[2-(2-imidazoline-2-yl)propane]disulfate dihydrate, 2,2′-azobis(2-methylpropionamidine)dihydrochloride, 2,2′-azobis[N-(2-carboxyethyl)-2-methylpropionamidine)]hydrate, 3-hydroxy-1,1-dimethylbutyl peroxyneodecanoate, α-cumylperoxy neodecanoate, 1,1,3,3-tetrabutyl peroxyneodecanoate, t-butyl peroxyneodecanoate, t-butyl peroxyneoheptanoate, t-butyl peroxypivalate, t-amyl peroxyneodecanoate, t-amyl peroxypivalate, di(2-ethylhexyl)peroxydicarbonate, di(secondary butyl)peroxydicarbonate, and azobiscyanovaleric acid. Further, for example, a reducing agent such as sodium sulfite, sodium hydrogen sulfite, or ascorbic acid may be used in combination with the radical polymerization initiator as a redox type initiator. A blending amount of the polymerization initiator is preferably 0.0005 to 0.005 mol with respect to 1 mol of a total amount of the monomers. With such a blending amount of the polymerization initiator, copolymerization of the respective monomers can efficiently proceed.

The polymerization initiator as it is may be mixed with the alkoxyalkyl (meth)acrylate of the Formula (1), the furfuryl (meth)acrylate of the Formula (2), if being used, a monomer which is copolymerizable with those components (another monomer, copolymerizable monomer; the same applies hereinafter), and a polymerization solvent, or alternatively a solution of the polymerization initiator obtained by being dissolved in another solvent in advance may be mixed with the monomers and the polymerization solvent. In the latter case, another solvent used to dissolve the polymerization initiator is not particularly limited as long as it can dissolve the polymerization initiator, but the same solvent as the polymerization solvent described above can be exemplified. Further, another solvent may be the same as or different from the polymerization solvent, but in consideration of easy control of polymerization, and the like, the same solvent as the polymerization solvent is preferably used. Further, in this case, a concentration of the polymerization initiator in another solvent is not particularly limited, but in consideration of easy mixing, and the like, the addition amount of the polymerization initiator is preferably 0.1 to 10 parts by weight and more preferably 0.5 to 5 parts by weight, with respect to 100 parts by weight of another solvent.

Further, in the case of using the polymerization initiator in the solution state, deaeration treatment may be performed in advance before adding a solution in which the monomers (alkoxyalkyl (meth)acrylate, furfuryl (meth)acrylate, and a copolymerizable monomer which is used as necessary) are dissolved in the polymerization solvent, to the polymerization initiator solution. For the deaeration treatment, for example, the solution may be bubbled with an inert gas such as nitrogen gas or argon gas for about 0.5 to 5 hours. In the deaeration treatment, the solution may be adjusted to about 30° C. to 80° C., preferably to a polymerization temperature in a polymerization step as described below.

Next, the monomer solution is heated to copolymerize the respective monomers. Here, as the copolymerization method, for example, a known polymerization method such as radical polymerization, anionic polymerization, or cationic polymerization can be adopted, and radical polymerization which facilitates production is preferably used.

The polymerization conditions are not particularly limited as long as the alkoxyalkyl (meth)acrylate of the Formula (1), the furfuryl (meth)acrylate of the Formula (2), and if being used, a monomer which is copolymerizable with those components (another monomer, copolymerizable monomer) can be copolymerized. Specifically, the copolymerization temperature is preferably 30 to 80° C. and more preferably 40° C. to 55° C. Further, the copolymerization time is preferably is 1 to 24 hours and more preferably 5 to 12 hours. Under such conditions, copolymerization of the respective monomers can efficiently proceed. Further, it is possible to effectively suppress or prevent gelation in the polymerization step and to achieve high production efficiency.

As necessary, a chain transfer agent, a polymerization rate-adjusting agent, a surfactant, and other additives may be appropriately used during the polymerization.

An atmosphere under which the polymerization reaction is carried out is not particularly limited, and the reaction can be carried out under an air atmosphere, an inert gas atmosphere such as nitrogen gas or argon gas, and the like. Further, during the polymerization reaction, the reaction solution may be stirred.

The polymer after polymerization can be purified by a general purification method such as a reprecipitation method (precipitation method), a dialysis method, an ultrafiltration method, or an extraction method.

The purified polymer can be dried by an arbitrary method such as freeze drying, vacuum drying, spray drying, or heat drying, but freeze drying or vacuum drying is preferred from the viewpoint that the physical properties of the polymer are less affected.

### [Polymer Substrate]

In the present invention, a coating layer containing the copolymer according to the present invention is formed on at least one surface of the polymer substrate. Herein, the coating layer is formed on at least a surface of the polymer substrate with which cells contact (for example, on which a liquid containing cells flows or cells are cultured). Further, it is not necessary to form the coating layer on an entire surface of the polymer substrate. The coating layer may be formed on a portion (a part) of the surface of the polymer substrate with which cells contact (for example, on which a liquid containing cells flows or cells are cultured). From the viewpoint of further improving cell adhesion (and further cell proliferation activity) and antithrombogenicity (particularly, in the case of using a blood sample), the coating layer is preferably formed on the entire surface of the polymer substrate at the side with which cells contact (for example, on which a liquid containing cells flows or cells are cultured).

Herein, a structure of the polymer substrate is not limited. In addition to the plane structure, the polymer substrate can be designed in various structures (forms) such as a structure in which a porous body is inserted, a hollow fiber structure, a porous membrane structure, a sponge structure, a flocculent (glass wool) structure. As described later, the cell culture substrate of the present invention can be suitably used in a bioreactor, particularly, a hollow fiber type bioreactor. Therefore, the polymer substrate preferably has hollow fibers and is more preferably a porous membrane formed of a plurality of hollow fibers. That is, according to the preferred embodiment of the present invention, the polymer substrate is a porous membrane. In the case where the polymer substrate is a porous membrane, an inner diameter (diameter) of the hollow fiber constituting the porous membrane is not particularly limited, but is preferably 50 to 1,000 μm, more preferably 100 to 500 μm, and particularly preferably about 150 to 350 μm. An outer diameter (diameter) of the hollow fiber constituting the porous membrane is not particularly limited, but is preferably 100 to 1,200 μm, more preferably 150 to 700 μm, and particularly preferably about 200 to 500 μm. A length of the hollow fiber constituting the porous membrane when the polymer substrate is a porous membrane is not particularly limited, but is preferably 50 to 900 mm, more preferably 100 to 700 mm, and particularly preferably about 150 to 500 mm. The number of the hollow fibers constituting the porous membrane when the polymer substrate is a porous membrane is not particularly limited, but is, for example, about 1,000 to 100,000, more preferably 3,000 to 50,000, and particularly preferably about 5,000 to 25,000. In an embodiment, the polymer substrate is configured by about 9,000 hollow fibers having an average length of about 295 mm, an average inner diameter of 215 μm, and an average outer diameter of 315 μm. Herein, the coating layer may be formed on the inner side or the outer side of the hollow fiber membrane, but is preferably formed on the inner (lumen) surface.

A method for producing a hollow fiber and a porous membrane is not particularly limited, and a known production method can be applied similarly or appropriately modified. For example, it is preferable that micro fine holes are formed on a wall of hollow fiber by a stretching method or a solid-liquid phase separation method.

A material constituting the polymer substrate is also not particularly limited. Specific examples thereof include a polyolefin resin such as polypropylene or polyethylene, a hydrophobic polymer material such as polystyrene, polysulfone, polyacrylonitrile, polytetrafluoroethylene, or cellulose acetate, and the like. Further, the polymer substrate may be produced by a semi-permeable, biocompatible polymer material such as a blend of polyamide, polyarylethersulfone, and polyvinylpyrrolidone (PA/PAES/PVP). Such a semi-permeable membrane allows transfer of nutrient, waste, and dissolved gas through the membrane between the extracapillary (EC) space of the hollow fiber and the intracapillary (IC) space of the hollow fiber. The molecule transfer characteristics of the hollow fiber membrane may be selected such that a metabolic waste product can pass through the membrane to be dispersed into a hollow fiber lumen and then removed therefrom, and at the same time, loss of an expensive reagent (such as a growth factor or cytokine) necessary for cell growth from the hollow fiber can be minimized. In a case where the polymer substrate is hollow fibers formed of PA/PAES/PVP, an outer layer of the hollow fiber may have an open pore structure with a certain surface roughness. An opening (diameter) of the pore is not particularly limited, but is in the range of about 0.5 to about 3 μm, and the number of pores on the outer surface of the hollow fiber may be in the range of about 10,000 to about 150,000 per 1 square millimeter (1 mm²). A thickness of the outer layer of the hollow fiber is not particularly limited, and for example, is in the range of about 1 to about 10 μm. The hollow fiber may have an additional layer (second layer) on the outer side, and at this time, the additional layer (second layer) preferably has a sponge structure having a thickness of about 1 to about 15 μm. The second layer having such a structure can serve as a support for the outer layer. Further, in this embodiment, the hollow fiber may have a further additional layer (third layer) at the outer side of the second layer. In this embodiment, the further additional layer (third layer) preferably has a finger-like structure. With the third layer having such a structure, mechanical stability is obtainable. Further, a high void volume with low resistance to membrane transfer of molecules can be provided. In this embodiment, during use, the finger-like voids are filled with a fluid and the fluid lowers resistance for diffusion and convection as compared with a matrix with a sponge-filled structure having a lower void volume. This third layer has a thickness of, preferably, about 20 to about 60 μm.

Further, the polymer substrate may have about 65% by weight to about 95% by weight of at least a hydrophobic polymer and about 5% by weight to about 35% by weight of at least a hydrophilic polymer. At this time, a total amount of the hydrophobic polymer and the hydrophilic polymer is 100% by weight. Here, the hydrophobic polymer is not particularly limited, and examples thereof include polyamide (PA), polyaramide (PAA), polyarylethersulfone (PAES), polyethersulphone (PES), polysulfone (PSU), polyarylsulphone (PASU), polycarbonate (PC), polyether, polyurethane (PUR), polyetherimide, and polyethersulphone; a mixture of polyarylethersulfone and polyamide; and the like. These hydrophobic polymers may be used singly or as a mixture of two or more kinds thereof. Further, the hydrophilic polymer is not particularly limited, and examples thereof include polyvinylpyrrolidone (PVP), polyethylene glycol (PEG), polyglycolmonoester, water soluble cellulosic derivatives, polysorbate, polyethylene-polypropylene oxide copolymers, and the like. These hydrophilic polymers may be used singly or as a mixture of two or more kinds thereof.

A method of forming a coating layer containing the copolymer according to the present invention on a surface of the polymer substrate is not particularly limited. For example, in a case where the surface of the polymer substrate has a flat dish (plate) structure, a method of applying a copolymer-containing solution obtained by dissolving the copolymer according to the present invention to a predetermined surface (for example, by adding to a well) and then drying coating film can be used. Further, for example, in a case where the polymer substrate is a hollow fiber or a porous membrane, a method of bringing a copolymer-containing solution obtained by dissolving the copolymer according to the present invention into contact with a cell contact portion of the hollow fiber (for example, by flowing on an inner surface (lumen) or an outer surface of the hollow fiber) and then drying coating film can be used. Incidentally, in a case where the polymer substrate is a porous membrane formed by a plurality of hollow fibers, coating with a copolymer-containing solution may be performed with respect to one hollow fiber and then the hollow fibers may be bundled, or a plurality of hollow fibers are bundled to produce a porous membrane and then the coating may be performed.

Herein, a solvent for dissolving the copolymer according to the present invention is not particularly limited as long as it can dissolve the copolymer according to the present invention. From the viewpoint of solubility of the copolymer, and the like, for example, aqueous solvents such as water, alcohols such as methanol or propanol, and polyethylene glycols; ketone-based solvents such as acetone; furan-based solvents such as tetrahydrofuran; and the like are exemplified. The solvent may be used singly or in the form of a mixture of two or more kinds thereof. Among these, in consideration of further improvement in solubility of the copolymer according to the present invention, the solvent is preferably methanol. A concentration of the copolymer in the copolymer-containing solution is not particularly limited. In consideration of the easy application to the substrate, the effects of reducing coating unevenness, and the like, the concentration thereof is preferably 0.0001 to 5% by weight and more preferably 0.001 to 1% by weight.

Further, a method of coating the copolymer is not particularly limited, and a conventionally known method such as filling, dip coating (immersion method), spraying, spin coating, dropping, doctor blade, brush coating, roll coater, air knife coating, curtain coating, wire bar coating, gravure coating, or mixed solution-impregnated sponge coating can be applied.

Further, conditions for forming the coating film of the copolymer are not particularly limited. For example, a contact time of the copolymer-containing solution and the polymer substrate (for example, a time for circulating the copolymer-containing solution to a lumen or an outer surface of the hollow fiber) is preferably 1 to 5 minutes and more preferably 1 to 3 minutes, in consideration of the easy formation of the coating film (thus coating layer), the effect of reducing coating unevenness, and the like. Further, a contact temperature of the copolymer-containing solution and the polymer substrate (for example, a temperature at which the copolymer-containing solution is circulated to a lumen or an outer surface of hollow fiber) is preferably 5 to 40° C. and more preferably 15 to 30° C., in consideration of the easy formation of the coating film (thus coating layer), the effect of reducing coating unevenness, and the like.

An amount of the copolymer-containing solution applied to a surface of the polymer substrate is not particularly limited, but is preferably such an amount that a thickness of the coating layer after drying is about 5 nm to 20 μm. Incidentally, in a case where such a thickness cannot be obtainable by single contact (application), a contact (application) step (or the application step and a drying step described later) may be repeated until a desired thickness is obtainable.

Next, by drying the coating film after the contact of the polymer substrate and the copolymer-containing solution, the coating layer (coating film) by the copolymer according to the present invention is formed on the surface of the polymer substrate. Herein, drying conditions are not particularly limited as long as the coating layer (coating film) of the copolymer according to the present invention can be formed. Specifically, a drying temperature is preferably 5 to 50° C. and more preferably 15 to 40° C. A drying step may be performed under a single condition or may be performed stepwise under different conditions. Further, the dry time is preferably 60 to 480 minutes and more preferably 120 to 360 minutes. Further, in a case where the polymer substrate is a porous membrane (hollow fiber membrane), the coating film may be dried by allowing a gas of 5 to 40° C. and more preferably 15 to 30° C. to continuously or gradually circulate on a surface of hollow fiber to which the copolymer-containing solution is applied. Herein, the gas is not particularly limited as long as it has no influence on the coating film (coating layer) and can dry the coating film. Specific examples thereof include air, an inert gas such as nitrogen gas or argon gas, and the like. Further, a circulation amount of the gas is not particularly limited as long as the coating film can be sufficiently dried. The circulation amount of the gas is preferably 5 to 150 L/min and more preferably 30 to 100 L/min.

According to such a method, the copolymer according to the present invention can be efficiently formed on the polymer substrate. Incidentally, depending on the type of cells to be adhered, the polymer substrate may be further treated with a cell adhesion factor such as fibronectin, laminin, or collagen. With such a treatment, adhesion of cells to the substrate surface and growth of cells can be further promoted. In a case where the polymer substrate is a porous membrane formed of a plurality of hollow fibers, the treatment with a cell adhesion factor may be performed with respect to one hollow fiber and then the hollow fibers may be bundled, or a plurality of hollow fibers are bundled to produce a porous membrane and then the treatment may be performed. Further, the treatment with a cell adhesion factor may be performed after the coating layer containing the copolymer according to the present invention is formed, before the coating layer containing the copolymer according to the present invention is formed, or at the same time the coating layer containing the copolymer according to the present invention is formed.

### <Bioreactor>

The cell culture substrate of the present invention exhibits cellular adhesiveness and antithrombogenicity with balance. Further, the cell culture substrate of the present invention has cell proliferation activity. Therefore, the cell culture substrate of the present invention can be suitably used in a bioreactor, particularly a bioreactor for culturing cells using a biological sample (particularly, a blood sample). That is, the present invention provides a bioreactor including the cell culture substrate of the present invention. Further, according to the preferred embodiment of the present invention, the present invention provides a bioreactor for culturing cells using a biological sample (particularly, a blood sample). Here, the bioreactor may be a plane type bioreactor or a hollow fiber type bioreactor, but is particularly preferably a hollow fiber type bioreactor. Therefore, in the following description, although a hollow fiber type bioreactor will be described as a preferred embodiment, the bioreactor of the present invention may be a plane type bioreactor, and in this case, the following embodiment can be appropriately changed and applied. Further, dimensional ratios in the drawings are exaggerated for the sake of explanatory convenience and may differ from actual ratios.

The bioreactor in which the cell culture substrate of the present invention can be suitably used is not particularly limited, but the cell culture substrate and the bioreactor of the present invention can be applied, for example, to cell culture/expansion systems described in JP 2010-523118 A (JP 5524824 B2)(WO 2008/124229 A2), JP 2013-524854 A (JP 6039547 B2) (WO 2011/140231 A1), JP 2013-507143 A (JP 5819835 B2) (WO 2011/045644 A1), JP 2013-176377 A (WO 2008/109674), JP 2015-526093 A (WO 2014/031666 A1), JP 2016-537001 A (WO 2015/073918 A1), JP 2017-509344 A (WO 2015/148704 A1), and the like; and Quantum Cell Expansion System manufactured by TERUMO BCT, INC. Conventionally, in the cell culture, facilities such as an incubator, a safety cabinet, and a clean room are separately needed, but the culture system as described above has all of those functions so that the facilities can be very simplified. Further, by controlling temperature or gas during the cell culture using the system as described above, a functionally closed system can be ensured and the cell culture can be performed automatically and in a closed environment.

Hereinafter, an embodiment of the bioreactor of the present invention will be described with reference to the drawings, but the present invention is not limited to the following embodiment.

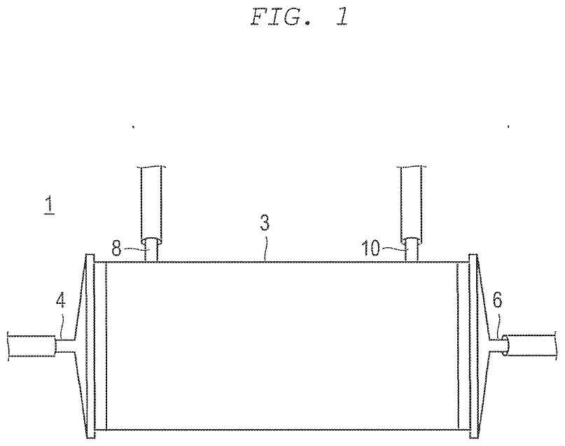

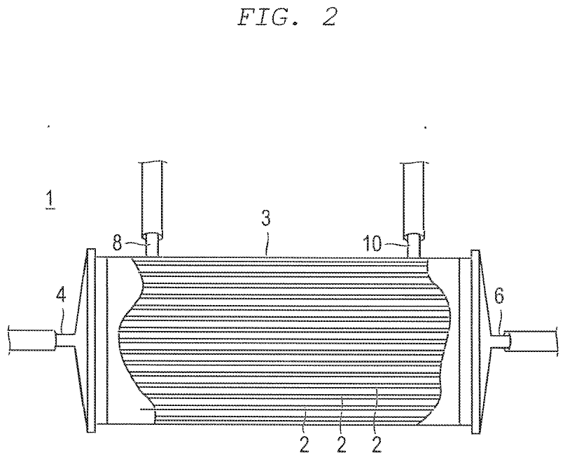

FIG. 1 is a partial side view illustrating an embodiment of a bioreactor (hollow fiber type bioreactor) of the present invention. Further, FIG. 2 is a partially cut-away side view of the bioreactor of FIG. 1. In FIGS. 1 and 2, a bioreactor 1 has a cell culture substrate 2 of the present invention provided in a cell culture chamber 3. The cell culture chamber 3 has four openings, that is, four ports (an inlet port 4, an outlet port 6, an inlet port 8, and an outlet port 10). Herein, a culture medium including cells flows to a hollow fiber intracapillary (IC) space of the cell culture substrate 2 in the cell culture chamber 3 through the inlet port 4, and discharged from the outlet port 6. According to this, cells are efficiently adhered (attached) to and cultured on the surface of the hollow fiber lumen. Meanwhile, a culture medium or gas (such as oxygen or carbon dioxide) flows to be in contact with a hollow fiber extracapillary (EC) space of the cell culture substrate 2 in the cell culture chamber 3 through the inlet port 8, and discharged from the outlet port 10. According to this, in the cell culture chamber 3, small molecules such as culture medium components flow into the hollow fibers or unnecessary components are discharged from the inside of the hollow fibers, and cells adhered onto the surface of the hollow fibers are cultured. Further, after culturing for a predetermined time, a liquid (for example, PBS) containing trypsin is introduced into the intracapillary (IC) space of the hollow fiber of the cell culture substrate 2 in the cell culture chamber 3 through the inlet port 4, and then is held for a predetermined time (for example, about 5 to 10 minutes). Next, a culture medium or an isotonic solution such as PBS flows in the intracapillary (IC) space of the hollow fiber of the cell culture substrate 2 in the cell culture chamber 3 through the inlet port 4 to apply a shear force to cells, the cells are released from the inner wall of the hollow fiber, and the cells are recovered from the bioreactor through the outlet port 6. Incidentally, although the cells are adhered to the intracapillary (IC) space of the hollow fiber in the above embodiment, the present invention is not limited to the above embodiment, and cells may be cultured in such a manner that a culture medium containing cells flows into the outlet port 10 from the inlet port 8, the cells are efficiently adhered (attached) to an outer surface of the hollow fiber, and the culture medium flows into the outlet port 6 from the inlet port 4 in an hollow fiber lumen. Further, the fluid from the inlet port 4 into the outlet port 6 may flow in either a co-current or counter-current direction with respect to flow of fluid into the outlet port 10 from the inlet port 8.

### [Use of Bioreactor]

As mentioned above, the bioreactor of the present invention includes a cell culture substrate excellent in cell adhesion (and further cell proliferation activity). Herein, cells which can be cultured in the bioreactor of the present invention may be adherent (scaffold-dependent) cells, non-adherent cells, or any combination thereof. In consideration of excellent cell adhesion (and further cell proliferation activity), the bioreactor of the present invention can be particularly suitably used in culturing of adherent (scaffold-dependent) cells. Herein, as the adherent (scaffold-dependent) cells, there are animal cells such as stem cells including mesenchymal stem cell (MSC) or the like, fibroblast cells, and the like. As mentioned above, attention has been paid to stem cells in development of regenerative medicine or drug discovery. Therefore, the bioreactor of the present invention can be suitably used in culturing of stem cells. That is, the present invention provides a method for culturing a stem cell using the bioreactor of the present invention. Herein, the method for culturing a stem cell is not particularly limited, and a general culturing method can be applied similarly or appropriately modified.

Further, as described above, the bioreactor of the present invention includes a cell culture substrate excellent in antithrombogenicity. Therefore, the bioreactor of the present invention can be particularly suitably used when cells are cultured using a biological sample (particularly, a blood sample). According to the embodiment, without unnecessary components in the biological sample (for example, platelets) being adhered to the substrate, target cells can be selectively and efficiently cultured.

### <Another Use>

The cell culture substrate of the present invention can exhibit cellular adhesiveness and antithrombogenicity with balance. Further, the cell culture substrate of the present invention has cell proliferation activity. Therefore, the cell culture substrate of the present invention can be suitably used as a biolumenal graft substrate, particularly, an implant (for example, artificial blood vessel) substrate which is placed in blood for a long time (depending on the cases, semipermanently). That is, the present invention provides a biolumenal graft including the cell culture substrate of the present invention. In the preferred embodiment of the present invention, there is provided an artificial blood vessel including the cell culture substrate of the present invention. In this embodiment, the biolumenal graft may have another configuration in addition to the cell culture substrate of the present invention (for example, as described later, may further have a stent) or may be the cell culture substrate of the present invention (may be configured by the cell culture substrate of the present invention). Further, according to the preferred embodiment of the present invention, the present invention provides an artificial blood vessel comprising the cell culture substrate of the present invention. According to the configuration, even when the artificial blood vessel is placed in blood for a long time (depending on the cases, semipermanently), thrombus formation (adhesion/attachment of platelets) can be effectively suppressed or prevented, and intimal formation can be effectively promoted. Herein, the artificial blood vessel is not limited to the following description, but there is an artificial blood vessel in which the copolymer according to the present invention is applied to or immersed in a conventionally known artificial blood vessel substrate. Further, in the biolumenal graft, a spring-shaped metal (stent part) called stent may be attached to the artificial blood vessel.

The biolumenal graft substrate of the present invention can be applied instead of a graft substrate (artificial blood vessel part) of a known biolumenal graft. For example, biolumenal graft substrate of the present invention may be applied instead of a graft fabric of JP 2008-505713 A (WO 2006/014592 A1), a circular thin film/thin-film tube of JP 2008-514309 A (WO 2006/037084 A1), lumen-shaped grafts of JP 2010-269161 A (WO 2004/002370 A1) and JP 2007-125415 A (WO 98/53761 A1), a biolumenal graft substrate of WO 2015/005105 A (US 2016/0135944 A1), and the like.

### EXAMPLES

The effects of the present invention will be described using the following examples and comparative examples. However, the technical scope of the present invention is not limited to only the following examples. Incidentally, in the following examples, operations were carried out at room temperature (25° C.) unless otherwise specified. In addition, unless otherwise specified, “%” and “part” mean “% by weight” and “parts by weight,” respectively.

### Production Example 1

### Synthesis of Copolymer (1)

To a 20-ml glass pressure-proof test tube, 0.9 g (0.0058 mol) of tetrahydrofurfuryl acrylate (THFA), 1.1 g (0.0085 mol) of methoxyethyl acrylate (MEA), and 3 g of methanol were added, and then nitrogen gas was bubbled for 10 seconds, thereby preparing a monomer solution (1). To this monomer solution (1), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining obtain a polymerization liquid (1). The resultant polymerization liquid (1) was added to 50 ml of hexane, and the precipitated polymer component was recovered and dried under reduced pressure, thereby obtaining a copolymer of tetrahydrofurfuryl acrylate and methoxyethyl acrylate (THFA:MEA=40:60 (molar ratio)) (copolymer (1)). The weight average molecular weight (Mw) of this copolymer (1) was measured to be 350,000.

### Production Example 2

### Synthesis of Copolymer (2)

To 20-ml glass pressure-proof test tube, 1.3 g (0.0083 mol) of tetrahydrofurfuryl acrylate (THFA), 0.7 g (0.0054 mol) of methoxyethyl acrylate (MEA), and 3 g of methanol were added, and then nitrogen gas was bubbled for 10 seconds, thereby preparing a monomer solution (2). To this monomer solution (2), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining obtain a polymerization liquid (2). The resultant polymerization liquid (2) was added to 50 ml of hexane, and the precipitated polymer component was recovered and dried under reduced pressure, thereby obtaining a copolymer of tetrahydrofurfuryl acrylate and methoxyethyl acrylate (THFA:MEA=60:40 (molar ratio)) (copolymer (2)). The weight average molecular weight (Mw) of this copolymer (2) was measured to be 320,000.

### Production Example 3

### Synthesis of Copolymer (3)

To 20-ml glass pressure-proof test tube, 1.65 g (0.0106 mol) of tetrahydrofurfuryl acrylate (THFA), 0.35 g (0.0027 mol) of methoxyethyl acrylate (MEA), and 3 g of methanol were added, and then nitrogen gas was bubbled for 10 seconds, thereby preparing a monomer solution (3). To this monomer solution (3), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining obtain a polymerization liquid (3). The resultant polymerization liquid (3) was added to 50 ml of hexane, and the precipitated polymer component was recovered and dried under reduced pressure, thereby obtaining a copolymer of tetrahydrofurfuryl acrylate and methoxyethyl acrylate (THFA:MEA=80:20 (molar ratio)) (copolymer (3)). The weight average molecular weight (Mw) of this copolymer (3) was measured to be 300,000.

### Production Example 4

### Synthesis of Copolymer (4)

To 20-ml glass pressure-proof test tube, 1.83 g (0.0117 mol) of tetrahydrofurfuryl acrylate (THFA), 0.17 g (0.0013 mol) of methoxyethyl acrylate (MEA), and 3 g of methanol were added, and then nitrogen gas was bubbled for 10 seconds, thereby preparing a monomer solution (4). To this monomer solution (4), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining obtain a polymerization liquid (4). The resultant polymerization liquid (4) was added to 50 ml of hexane, and the precipitated polymer component was recovered and dried under reduced pressure, thereby obtaining a copolymer of tetrahydrofurfuryl acrylate and methoxyethyl acrylate (THFA:MEA=90:10 (molar ratio)) (copolymer (4)). The weight average molecular weight (Mw) of this copolymer (4) was measured to be 310,000.

### Production Example 5

### Synthesis of THFA Polymer (5)

To 20-ml glass pressure-proof test tube, 2.00 g (0.0128 mol) of tetrahydrofurfuryl acrylate (THFA) and 3 g of methanol were added, and then nitrogen gas was bubbled for 10 seconds, thereby preparing a monomer solution (5). To this monomer solution (5), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining obtain a polymerization liquid (5). The resultant polymerization liquid (5) was added to 50 ml of hexane, and the precipitated polymer component was recovered and dried under reduced pressure, thereby obtaining a homopolymer of tetrahydrofurfuryl acrylate (THFA polymer (5)). The weight average molecular weight (Mw) of this THFA polymer (5) was measured to be 290,000.

### Production Example 6

### Synthesis of Copolymer (6)

To 20-ml glass pressure-proof test tube, 0.24 g (0.0015 mol) of tetrahydrofurfuryl acrylate (THFA), 1.76 g (0.0135 mol) of methoxyethyl acrylate (MEA), and 3 g of methanol were added, and then nitrogen gas was bubbled for 10 seconds, thereby preparing a monomer solution (6). To this monomer solution (6), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining obtain a polymerization liquid (6). The resultant polymerization liquid (6) was added to 50 ml of hexane, and the precipitated polymer component was recovered and dried under reduced pressure, thereby obtaining a copolymer of tetrahydrofurfuryl acrylate and methoxyethyl acrylate (THFA:MEA=10:90 (molar ratio)) (copolymer (6)). The weight average molecular weight (Mw) of this copolymer (6) was measured to be 370,000.

### Production Example 7

### Synthesis of Copolymer (7)

To 20-ml glass pressure-proof test tube, 0.46 g (0.0029 mol) of tetrahydrofurfuryl acrylate (THFA), 1.54 g (0.0118 mol) of methoxyethyl acrylate (MEA), and 3 g of methanol were added, and then nitrogen gas was bubbled for 10 seconds, thereby preparing a monomer solution (7). To this monomer solution (7), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours. The polymerization liquid was added to 50 ml of hexane, and the precipitated polymer component was recovered and dried under reduced pressure, thereby obtaining a copolymer of tetrahydrofurfuryl acrylate and methoxyethyl acrylate (THFA:MEA=20:80 (molar ratio)) (copolymer (7)). The weight average molecular weight (Mw) of this copolymer (7) was measured to be 375,000.

### Production Example 8

### Synthesis of MEA Polymer (8)

To 20-ml glass pressure-proof test tube, 2.0 g (0.0154 mol) of methoxyethyl acrylate (MEA) and 3 g of methanol were added, and then nitrogen gas was bubbled for 10 seconds, thereby preparing a monomer solution (8). To this monomer solution (8), 0.004 g (0.013 mmol) of 2,2′-azobis(4-methoxy-2,4-dimethylvaleronitrile) as a polymerization initiator was added, and the resultant mixture was heated in a heat block set at 45° C. for 6 hours, to perform polymerization reaction, thereby obtaining obtain a polymerization liquid (8). The resultant polymerization liquid (8) was added to 50 ml of hexane, and the precipitated polymer component was recovered and dried under reduced pressure, thereby obtaining a homopolymer of methoxyethyl acrylate (MEA polymer (8)). The weight average molecular weight (Mw) of this MEA polymer (8) was measured to be 380,000.

### Example 1: Coating to Cell Culture Dish

The copolymer (1) obtained in Production Example 1 was dissolved in methanol to have a concentration of 0.05% by weight, thereby producing a coating liquid (1). 25 μL of the coating liquid (1) was added to each well of commercially available 96-well tissue culture polystyrene dish (without a plasma treatment, manufactured by FALCON, trade name: Non-Tissue Culture Treated Plate, 96 Well, Flat Bottom with Low Evaporation Lid) and dried at 20° C. for 300 minutes to produce a polymer coating film (dry thickness: 0.3 μm), thereby obtaining a cell culture dish (1).

### Examples 2 to 4

### Coating to Cell Culture Dish

A polymer coating film was produced on the well surface according to the similar method to Example 1, except that, in Example 1, each of the copolymers (2) to (4) was used instead of the copolymer (1), thereby obtaining cell culture dishes (2) to (4).

### Comparative Examples 1 to 5

### Coating to Cell Culture Dish

A polymer coating film was produced on the well surface according to the similar method to Example 1, except that, in Example 1, each of the THFA polymer (5), the copolymers (6) and (7), and the MEA polymer (8) was used instead of the copolymer (1), thereby obtaining comparative cell culture dishes (1) to (4). Further, a commercially available 96-well tissue culture polystyrene dish (without a plasma treatment and a polymer coating film, manufactured by FALCON, trade name: Non-Tissue Culture Treated Plate, 96 Well, Flat Bottom with Low Evaporation Lid) was used as a comparative cell culture dish (5).

### Examples 5 to 8 and Comparative Examples 6 to 10

### Cell Culture and Measurement of Adhesion Activity

By the following method, cells were cultured using the cell culture dishes (1) to (4) and the comparative cell culture dishes (1) to (5) obtained in Examples 1 to 4 and Comparative Examples 1 to 5, and the cell adhesion activity (cell adhesion) was evaluated. Incidentally, as the cells, human adipose tissue-derived mesenchymal stem cells (Lonza, Walkersville, Md., U.S.A.) were used. The donor was a 22-year-old man and expressed CD13, CD29, CD44, CD73, CD90, CD105, CD166 90%, CD14, CD31, and CD45≤5%.

The human adipose tissue-derived mesenchymal stem cells were seeded on each well of the cell culture dishes (1) to (5) and the comparative cell culture dishes (1) to (5) to be 2×10³ cells/well, and then cultured for one day in Mesenchymal Stem Cell Growth Medium 2 (PromoCell GmbH, Bedford, Mass., U.S.A.) in an incubator under humidified conditions at 37° C. in the presence of 5% CO₂. After the completion of culture, the culture solution was exchanged with Mesenchymal Stem Cell Growth Medium 2 containing 10% WST-1 (Premix WST-1 Cell Proliferation Assay System, Takara Bio Inc., Shiga, Japan) and then incubated for about 4 hours under normal pressure (37° C., 5% CO₂) under humidified conditions. An absorbance (450 nm, comparison 600 nm) of the culture solution was measured by a microplate reader and regarded as cell adhesion activity. Results are presented in the following Table 1. In the following Table 1, a proportion of absorbance of the culture solution cultured in each culture dish in a case where the absorbance the culture solution cultured in the comparative cell culture dish (1) (Comparative Example 6) is regarded as “1.00” is shown as a proportion of adhesion activity.

TABLE 1

| | | | | | Ratio of |
|---|---|---|---|---|---|
| | Polymer | | | | adhesion |
| | | | Monomer | Cell adhesion | activity (to |
| | | Monomer | composition | activity | Comparative |
| | | type | (molar ratio) | (AbS₄₅₀) | Example 6) |
| Example 5 | Copolymer (1) | THFA-MEA | 40:60 | 0.219 | 1.090 |
| Example 6 | Copolymer (2) | THFA-MEA | 60:40 | 0.219 | 1.090 |
| Example 7 | Copolymer (3) | THFA-MEA | 80:20 | 0.218 | 1.085 |
| Example 8 | Copolymer (4) | THFA-MEA | 90:10 | 0.225 | 1.119 |
| Comparative | THFA polymer (5) | THFA | 100:0 | 0.201 | 1.000 |
| Example 6 | | | | | |
| Comparative | Copolymer (6) | THFA-MEA | 10:90 | 0.143 | 0.711 |
| Example 7 | | | | | |
| Comparative | Copolymer (7) | THFA-MEA | 20:80 | 0.186 | 0.925 |
| Example 8 | | | | | |
| Comparative | MEA polymer (8) | MEA | 0:100 | 0.111 | 0.552 |
| Example 9 | | | | | |

| | | | |
|---|---|---|---|
| Comparative | — | 0.067 | 0.333 |
| Example 10 | (Non-coating with polymer) | | |

From the comparison between Comparative Example 6 and Comparative Example 9 in the Table 1, it is noted that the comparative cell culture dish (4) having the polymer coating film of the MEA polymer (8) formed thereon has only a little more than 50% of cell adhesion activity as compared to the comparative cell culture dish (1) having the polymer coating film of the THFA polymer (5) formed thereon. On the other hand, the cell culture dishes (1) to (4) having the polymer coating films of the copolymers (1) to (4) according to the present invention having THFA and MEA at a specific composition are significantly excellent in cellular adhesiveness, as compared to the comparative cell culture dish (1) having the polymer coating film of the THFA polymer (5) formed thereon. Herein, in view that it is generally considered that cellular adhesiveness of the coating film of the copolymer obtained by copolymerization of tetrahydrofurfuryl acrylate with methoxyethyl acrylate inferior in cellular adhesiveness is degraded as compared to a coating film of a homopolymer of tetrahydrofurfuryl acrylate, the above-described result is very surprising.

### Example 9

### Production of Polymer-Coated Test Piece

The copolymer (1) obtained in the Production Example 1 was dissolved in methanol to have a concentration of 0.5% by weight, thereby producing a coating liquid (5). A polyethylene terephthalate sheet (width 1 cm×length 10 cm×thickness 100 μm) was immersed in this coating liquid (5) at 25° C. for 2 minutes (dip coating), and then dried at 25° C. for 360 minutes, thereby producing a polymer coated-test piece (1) (dry thickness of the polymer coating film: 0.5 μm).

### Examples 10 to 12

### Production of Polymer-Coated Test Piece

Polymer coated-test pieces (2) to (4) were produced according to the same method as in Example 9, except that in Example 9, each of the copolymers (2) to (4) was used instead of the copolymer (1).

### Comparative Examples 11 to 15

### Production of Polymer-Coated Test Piece

Comparative polymer coated-test pieces (1) to (4) were produced according to the same method as in Example 9, except that in Example 9, each of the THFA polymer (5), the copolymers (6) and (7), and the MEA polymer (8) was used instead of the copolymer (1). Further, a polyethylene terephtha late sheet (width 1 cm×length 10 cm×thickness 100 μm, without a polymer coating film) was provided as a comparative test piece (5).

### Examples 13 to 16 and Comparative Examples 16 to 20

### Platelet Adhesion Test (Antithrombogenicity Evaluation)

By the following method, a platelet adhesion test was performed using the polymer coated-test pieces (1) to (4) and the comparative polymer coated-test pieces (1) to (5) obtained in Examples 9 to 12 and Comparative Examples 11 to 15, to evaluate antithrombogenicity. An aqueous solution of 3.8 (w/v) % sodium citrate (anticoagulant) was added to pig fresh blood to obtain anticoagulated pig fresh blood. At the time, the blood and the sodium citrate aqueous solution were mixed such that a mixing ratio (volume ratio) thereof would be 9:1. The anticoagulated pig fresh blood was subjected to separation by centrifugation (1500 rpm) at 22° C. for 10 minutes, to obtain platelet-rich plasma (PRP). This PRP was filled in each of cylindrical containers having the polymer coated-test pieces (1) to (4), the comparative polymer coated-test pieces (1) to (4), and the comparative test piece (5) attached onto the bottom surfaces, and then the containers were left to stand still at room temperature (25° C.) for 5 hours. After a predetermined time elapsed, each test piece was washed with saline and then fixed with 1 (w/v) % glutaraldehyde saline. Each test piece was left to stand still at room temperature (25° C.) for 10 hours and then was washed with distilled water. Each test piece was dried under reduced pressure and subjected to vapor deposition of platinum, and then was observed with a scanning electron microscope (SEM) and captured (1000-fold magnification, five fields). From the photograph, the number of platelets attached (adhered) to the surface of each test piece was counted. Results are presented in the following Table 2. Incidentally, in the following Table 2, regarding the number of platelets, the total of platelets at 1000-fold magnification and five fields was calculated.

TABLE 2

| | Polymer | | | (Non- |
|---|---|---|---|---|
| | | | Monomer | coating |
| | | Monomer | composition | with |
| | | type | (molar ratio) | polymer) |
| Example 13 | Copolymer (1) | THFA-MEA | 40:60 | 3 |
| Example 14 | Copolymer (2) | THFA-MEA | 60:40 | 4 |
| Example 15 | Copolymer (3) | THFA-MEA | 80:20 | 7 |
| Example 16 | Copolymer (4) | THFA-MEA | 90:10 | 9 |
| Comparative | THFA polymer | THFA | 100:0 | 62 |
| Example 16 | (5) | | | |
| Comparative | Copolymer (6) | THFA-MEA | 10:90 | 1 |
| Example 17 | | | | |
| Comparative | Copolymer (7) | THFA-MEA | 20:80 | 1 |
| Example 18 | | | | |
| Comparative | MEA polymer (8) | MEA | 0:100 | 1 |
| Example 19 | | | | |

| | | |
|---|---|---|
| Comparative | — | 753 |
| Example 20 | (Non-coating with polymer) | |

From the result of the above Table 2, it is found that the polymer coated-test pieces (1) to (4) having polymer coating films of the copolymers (1) to (4) of Production Examples 1 to 4 show the slightly large number of attached platelets and slightly degraded antithrombogenicity, as compared to the comparative polymer coated-test pieces (4), (2), and (3) having polymer coating films of the MEA polymer (8) excellent in antithrombogenicity and of the THFA-MEA copolymers (6) and (7) that are out of the composition according to the present invention. On the other hand, as described above, as shown in the Table 1, the cell culture dishes (1) to (4) according to the present invention show significantly excellent cellular adhesiveness, as compared to the comparative cell culture dishes (4), (2), and (3) having the polymer coating films of the MEA polymer (8) and the THFA-MEA copolymers (6) and (7). Taking in consideration of those results in combination, it is considered that the cell culture substrate of the present invention can achieve both cellular adhesiveness and antithrombogenicity with better balance, as compared to the THFA polymer excellent in cellular adhesiveness, the MEA polymer excellent in antithrombogenicity, and the THFA-MEA copolymers that are out of the composition according to the present invention.

### REFERENCE SIGNS LIST

1 BIOREACTOR

2 CELL CULTURE SUBSTRATE

3 CELL CULTURE CHAMBER

4, 8 INLET PORT

6, 10 OUTLET PORT

1. A cell culture substrate comprising a coating layer on at least one side of a polymer substrate, wherein the coating layer includes a copolymer comprising 5 to 65% by mole of a structural unit (1) derived from alkoxyalkyl (meth)acrylate of following Formula (1) and 95 to 35% by mole of a structural unit (2) derived from furfuryl (meth)acrylate of following Formula (2) (a total of the structural unit (1) and the structural unit (2) is 100% by mole): 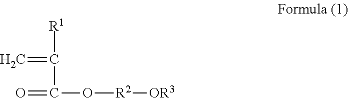
wherein R¹ represents a hydrogen atom or a methyl group, R² represents an alkylene group having 2 or 3 carbon atoms, and R³ represents an alkyl group having 1 to 3 carbon atoms; 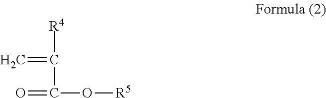
wherein R⁴ represents a hydrogen atom or a methyl group, and R⁵ represents a group represented by following Formula (2-1) or following Formula (2-2): 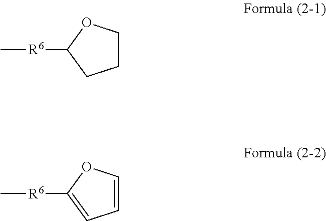
wherein R⁶ represents an alkylene group having 1 to 3 carbon atoms.

2. The cell culture substrate according to claim 1, wherein the copolymer is a copolymer comprising 10 to 60% by mole of the structural unit (1) derived from alkoxyalkyl (meth)acrylate of the Formula (1) and 40 to 90% by mole of the structural unit (2) derived from furfuryl (meth)acrylate of the Formula (2) (the total of the structural unit (1) and the structural unit (2) is 100% by mole).

3. The cell culture substrate according to claim 1, wherein the copolymer is composed of the structural unit (1) and the structural unit (2).

4. The cell culture substrate according to claim 1, wherein the polymer substrate is a porous membrane.

5. A bioreactor comprising the cell culture substrate according to claim 1.

6. A method for culturing a stem cell using the bioreactor according to claim 5.

7. The cell culture substrate according to claim 2, wherein the copolymer is composed of the structural unit (1) and the structural unit (2).

8. The cell culture substrate according to claim 2, wherein the polymer substrate is a porous membrane.

9. A bioreactor comprising the cell culture substrate according to claim 2.
